(19) United States
(12) Patent Application Publication
(10) Pub. No.: US 2008/0287429 A1
(43) Pub. Date: Nov. 20, 2008

(54) Dissolution of Arterial Cholesterol Plaques by Pharmacologically Induced Elevation of Endogenous Bile Salts

(75) Inventors: Filiberto P. Zadini, Camarillo, CA (US); Giorgio C. Zadini, Camarillo, CA (US)

Correspondence Address: MCDERMOTT WILL & EMERY LLP, 18191 VON KARMAN AVE., SUITE 500, IRVINE, CA, 92612-7108, US

(73) Assignee: Z & Z Medical Holdings, Inc., Laguna Niguel, CA (US)

(21) Appl. No.: 12/121,769

(22) Filed: May 15, 2008

Related U.S. Application Data
(60) Provisional application No. 60/930,410, filed on May 15, 2007.

(51) Int. Cl. A61K 31/536; A61K 31/4164; A61K 31/035; A61K 31/426; A61P 9/10; A61K 31/427; A61K 31/505; A61K 31/47

(52) U.S. Cl. 514/230.5; 514/397; 514/744; 514/369; 514/269; 514/307; 514/365

(57) ABSTRACT

A group of pharmaceutical substances induce elevation of endogenous bile salts and acids via different mechanisms. The elevated circulating bile salts exert a beneficial effect in atherosclerosis by acting both as atherolytic and antiatherogenic agents. The result of the elevated circulating endogenous bile salt is the dissolution of cholesterol/lipidic aggregates of the atherosclerotic plaques.

### RELATED APPLICATIONS

This application claims priority to U.S. Provisional Patent Application No. 60/930,410, filed on May 15, 2007, and entitled “Dissolution of Arterial Cholesterol Plaques by Pharmaceutically Induced Elevation of Endogenous Biliary Salts,” the contents of which are hereby incorporated by reference in their entirety.

### FIELD OF THE INVENTION

Some embodiments of the invention comprise pharmaceutical compounds or formulations useful in atherosclerotic plaque treatments in mammals. Certain embodiments described herein comprise pharmaceutical compounds or formulations effective to divert endogenous bile acids, bile salts, their precursors, and/or their derivatives from the enterohepatic circulation of a mammal to the systemic circulation such that the diverted bile acids, bile salts, their precursors, and/or their derivatives are present in the systemic circulation in concentrations effective to emulsify and dissolve components of an atherosclerotic plaque, either in a plaque or in circulation, resulting in regression in a size of the plaque and/or inhibition of atherogenesis.

### BACKGROUND OF THE INVENTION

Atherosclerosis is a pathological condition responsible for mortality and morbidity in humans. No known pharmaceutical compound has been shown in studies to unequivocally reduce preexisting atherosclerotic lesions to the point that clinical benefits would ensue.

Cardiovascular disease is a leading cause of death in the human population. This is especially true in developed countries, where the increasing incidence of obesity is considered to be the major contributing factor to cardiovascular and related diseases. For example, the incidence of heart disease as a cause of death was 12.4% in all World Health Organization States, whereas in the U.S., heart attacks account for nearly 30% of deaths. In addition, other disease states related to or exacerbated by impairment of cardiovascular function make cardiovascular diseases the single greatest contributor to death and disability.

The underlying issue in cardiovascular disease is the development of atherosclerosis, a disease that affects vessels of the arterial circulation. It is characterized as a chronic inflammatory response in the walls of blood vessels, in part due to deposition of lipoproteins, in particular low density lipoproteins (LDLs), as well as infiltration by macrophages. Atherosclerosis is known to begin during childhood with the rate of progression dependent on a variety of factors including diet, exercise, and genetic predisposition.

The earliest morphologically identifiable stage of plaque development is termed a fatty streak, which in fact is an accumulation of macrophages that have ingested oxidized LDL in the vessel wall, giving them the appearance of fat in the muscular tissue that forms the vessel wall. These macrophages ingest oxidized LDL in the plaque, accumulate numerous cytoplasmic vesicles, and are known as foam cells. Over time the fatty streak evolves to become an established plaque characterized by further accumulation of macrophages and the local accumulation of an inflammatory infiltrate. Eventually foam cells die, releasing their contents into the plaque, which further exacerbates the inflammatory reaction. In addition, cytokines released by damaged endothelial cells lead to smooth muscle proliferation and migration from the vessel media to the intima, leading to the development of a fibrous capsule that covers the plaque. Over time, calcification at the margins of the plaque can occur.

It has been known for some time that over time that progressive enlargement of atherosclerotic plaques eventually leads to a narrowing of the lumen of afflicted vessels. Traditionally, narrowing of 75% or greater has been considered clinically significant. However, more recently it has been discovered that events such as heart attacks can occur even when there is no sign of significant narrowing of vessels, due to the inherent instability of some plaques.

It is now known that plaques can be structurally unstable, and spontaneously rupture. When a plaque ruptures, tissue fragments and plaque contents are released into the lumen of the blood vessel, resulting in a clotting response. While the clot is effective to cover and stabilize the rupture, it intrudes into the lumen of the vessel, reducing luminal diameter, and obstructing blood flow, thus creating a stenotic region. If the compromise to flow is significant, for example where the clot completely or nearly completely occludes the lumen, ischemia can occur in tissues downs stream from the site of the blockage. Where the vessel is a coronary artery, this can lead to a myocardial infarction. Should the blockage occur in a cerebral artery stroke is possible. Significantly, the majority of fatal events occur from ruptures in areas where there is little prior narrowing, although it is recognized that over time repeated ruptures of plaques will lead to stenosis, and eventually downstream ischemia, with the same clinical outcome.

Because of the risk posed by unstable plaque, there is now a recognized need to detect atherosclerotic plaque, and in particular soft, or vulnerable plaque, prior to the patient becoming symptomatic. Earlier detection of vulnerable plaque can be especially useful in order to begin a course of treatment that can reduce the risk of a sudden ischemic event due to plaque rupture, or due to the gradual development of stenotic regions in a vessel as can occur over time, or to reopen areas of vessel that have become substantially occluded. Typically, treatment of stenosis in sensitive areas such as the heart or the brain has been accomplished by angioplasty techniques. Maintaining patency of vessels has become easier with the advent of vascular stent devices.

In the past, detection and diagnosis of atherosclerosis has been difficult. For example, according to data in the U.S. from 2004, the first symptom of cardiovascular disease in over half of those so diagnosed, is heart attack or sudden death. Unfortunately, by the time obvious symptoms arose, the disease is usually quite advanced with the result that treatment options and clinical outcome can be limited. The recognition of contributing factors such as the effect of cholesterol intake, obesity, and smoking, has led to an awareness of the benefit of preventative lifestyle choices in reducing the risk of developing atherosclerosis.

More recently, advances have also been made in both the diagnosis and treatment of cardiovascular disease. For example, 64 slice CT technology now makes it possible to evaluate the extent cardiovascular disease through detection of calcifications in vessels. In addition, CT protocols are also available that make it possible to visualize vulnerable plaque. Thus, it is becoming easier to detect atherosclerosis at earlier and earlier stages, providing an ever increasing window of opportunity to treat the disease at as early a stage as possible.

There are known medications, such as statins, which significantly lower serum cholesterol, and lowering serum cholesterol indeed translates into reduced probability of new plaque formation. But lowering serum cholesterol with such drugs does not translate into clinically significant reductions in the size of preexisting plaques. Nor does lowering serum cholesterol translate into clinically significant reductions in health risks, such as plaque rupture and thrombosis, posed by atherosclerotic plaques.

While prior art treatments can be effective to deal with some of the factors that contribute to the development of atherosclerotic plaque (e.g., use of statins to reduce cholesterol levels), or to open occlude vessel (e.g., angioplasty and vascular stents) there remains a need for effective ways regress existing plaque size and burden in patients.

Applicants have disclosed in U.S. Provisional Patent Application No. 60/739,143, filed on Nov. 22, 2005; U.S. patent application Ser. No. 11/373,943, filed on Mar. 13, 2006; U.S. patent application Ser. No. 11/384,150, filed on Mar. 17, 2006; international patent application PCT/US 2006/044619, filed on Nov. 16, 2006; and U.S. patent application Ser. No. 11/649,062, filed on Jan. 3, 2007, the contents of each of which are hereby incorporated by reference in their entireties, a class of physiological emulsifiers, namely bile salts, bile acids, their precursors and/or derivatives, that emulsify and dissolve atherosclerotic plaques. Applicants have experimentally demonstrated that such emulsifiers penetrate the fibrous cap of atherosclerotic plaques and emulsify and dissolve atherosclerotic plaques and components of atherosclerotic plaques, such as lipids, e.g., cholesterol, either in plaques or in circulation.

It is known that, due to the enterohepatic circulation, endogenous bile salts are not normally present in the systemic circulation of a mammal in concentrations effective to emulsify and dissolve atherosclerotic plaques. Applicants have disclosed in the above mentioned patent applications routes for administering exogenous bile salts, bile acids, their precursors, and their derivatives that bypass the enterohepatic circulation, rendering them bioavailable in the systemic circulation. Such routes include: Intravenous; Intradermal/transdermal; Oral Mucous membrane, such as sublingual; Subcutaneous via injection for prompt or slow release; Rectal, for instance in the form of a suppository; Intramuscular for prompt or slow release, such as in a depo form; Inhalation, such as in a form of inhaled microcrystals or aerosol; vaginal; intraperitoneal; and others.

### SUMMARY OF THE INVENTION

Some embodiments comprise pharmaceutical compounds or formulations useful in atherosclerotic plaque treatments in mammals. The pharmaceutical compounds and formulations of some embodiments are effective to make bioavailable, in the systemic circulation of a mammal, endogenous bile salts, bile acids, precursors of bile salts and acids, and derivatives of bile salts and acids by producing their diversion from the enterohepatic circulation to the systemic circulation in concentrations effective to emulsify and/or dissolve atherosclerotic plaques and plaque components, especially lipids such as cholesterol, either in a plaque or in circulation, thereby providing an atherolytic or antiatherogenic effect.

Accordingly, in some embodiments, there is provided a pharmaceutical formulation, for treating atherosclerosis in a mammal, comprising a combination of at least two of (i)-(vii): (i) ketoconazole or a pharmaceutically acceptable salt, conjugate, hydrate, solvate, polymorph, or mixture thereof; (ii) trichloroethylene or a pharmaceutically acceptable salt, conjugate, hydrate, solvate, polymorph, or mixture thereof; (iii) troglitazone or a pharmaceutically acceptable salt, conjugate, hydrate, solvate, polymorph, or mixture thereof; (iv) bosentan or a pharmaceutically acceptable salt, conjugate, hydrate, solvate, polymorph, or mixture thereof; (v) saquinavir or a pharmaceutically acceptable salt, conjugate, hydrate, solvate, polymorph, or mixture thereof; (vi) ritonavir or a pharmaceutically acceptable salt, conjugate, hydrate, solvate, polymorph, or mixture thereof; (vii) efavirenz or a pharmaceutically acceptable salt, conjugate, hydrate, solvate, polymorph, or mixture thereof; wherein the combination is in an amount effective to result in an amount of increased diversion of a bile acid, from an enterohepatic circulation to the systemic circulation of the mammal, sufficient to result in an amount of emulsification of an atherosclerotic plaque in an artery of the mammal sufficient to result in regression of the plaque.

In some embodiments, ketoconazole, trichloroethyelen, troglitazone, bosentan, saquinovir, ritanovir, and efavirenz may be used in combination, each at individual doses lower than doses for each of ketoconazole, trichloroethyelene, troglitazone, bosentan, saquinovir, ritanovir, and efavirenz alone.

In some embodiments, the combination comprises ketoconazole or a pharmaceutically acceptable salt, conjugate, hydrate, solvate, polymorph, or mixture thereof in an amount of 200 mg or greater. In some embodiments, the combination comprises trichloroethylene or a pharmaceutically acceptable salt, conjugate, hydrate, solvate, polymorph, or mixture thereof in an amount of 1 mg or greater. In some embodiments, the combination comprises troglitazone or a pharmaceutically acceptable salt, conjugate, hydrate, solvate, polymorph, or mixture thereof in an amount of 200 mg or greater. In some embodiments, the combination comprises bosentan or a pharmaceutically acceptable salt, conjugate, hydrate, solvate, polymorph, or mixture thereof in an amount of 30 mg or greater. In some embodiments, the combination comprises saquinavir or a pharmaceutically acceptable salt, conjugate, hydrate, solvate, polymorph, or mixture thereof in an amount of 1000 mg or greater. In some embodiments, the combination comprises ritonavir or a pharmaceutically acceptable salt, conjugate, hydrate, solvate, polymorph, or mixture thereof in an amount of 400 mg or greater. In some embodiments, the combination comprises efavirenz or a pharmaceutically acceptable salt, conjugate, hydrate, solvate, polymorph, or mixture thereof in an amount of 200 mg or greater.

In addition, some embodiments provide a method, of treating atherosclerosis in a mammal, comprising administering to a mammal a pharmaceutical formulation in an amount effective to result in an amount of increased diversion of a bile acid, from an enterohepatic circulation to the systemic circulation of the mammal, sufficient to result in an amount of emulsification of an atherosclerotic plaque in an artery of the mammal sufficient to result in regression of the plaque.

In some embodiments, the formulation comprises an active ingredient consisting essentially of at least one of: (i) ketoconazole or a pharmaceutically acceptable salt, conjugate, hydrate, solvate, polymorph, or mixture thereof; (ii) trichloroethylene or a pharmaceutically acceptable salt, conjugate, hydrate, solvate, polymorph, or mixture thereof; (iii) troglitazone or a pharmaceutically acceptable salt, conjugate, hydrate, solvate, polymorph, or mixture thereof; (iv) bosentan or a pharmaceutically acceptable salt, conjugate, hydrate, solvate, polymorph, or mixture thereof; (v) saquinavir or a pharmaceutically acceptable salt, conjugate, hydrate, solvate, polymorph, or mixture thereof; (vi) ritonavir or a pharmaceutically acceptable salt, conjugate, hydrate, solvate, polymorph, or mixture thereof; and (vii) efavirenz or a pharmaceutically acceptable salt, conjugate, hydrate, solvate, polymorph, or mixture thereof.

In some embodiments, the administering results in a total serum bile acid concentration in the systemic circulation of greater than about 60 μM. In some embodiments, the administering results in a total serum bile acid concentration in the systemic circulation of about 100 μM to about 300 μM. In some embodiments, the administering results in a total serum bile acid concentration in the systemic circulation of above about 300 μM. In some embodiments, the administering results in a total serum bile acid concentration in the systemic circulation of above about 600 μM. In some embodiments, the bile acid comprises deoxycholic acid.

In some embodiments, the formulation comprises ketoconazole or a pharmaceutically acceptable salt, conjugate, hydrate, solvate, polymorph, or mixture thereof, and wherein the ketoconazole is administered to the mammal at a dose of greater than 600 mg/day. In some embodiments, the formulation comprises ketoconazole or a pharmaceutically acceptable salt, conjugate, hydrate, solvate, polymorph, or mixture thereof, and the ketoconazole is administered orally to the mammal at a dose of greater than 600 mg/day for at least 7 days. In some embodiments, the formulation comprises trichloroethylene or a pharmaceutically acceptable salt, conjugate, hydrate, solvate, polymorph, or mixture thereof, and the trichloroethylene is administered in a single or divided dose of greater than 135 mg/kg. In some embodiments, the formulation comprises trichloroethylene or a pharmaceutically acceptable salt, conjugate, hydrate, solvate, polymorph, or mixture thereof, and the trichloroethylene is administered in a single or divided dose of greater than 1 mg/kg/day for at least 7 days. In some embodiments, the formulation comprises troglitazone or a pharmaceutically acceptable salt, conjugate, hydrate, solvate, polymorph, or mixture thereof, and the troglitazone is administered in a single or divided dose of greater than 30 mg/kg/day. In some embodiments, the formulation comprises troglitazone or a pharmaceutically acceptable salt, conjugate, hydrate, solvate, polymorph, or mixture thereof, and the troglitazone is administered in a single or divided dose of greater than 500 mg/day for at least 28 days. In some embodiments, the formulation comprises bosentan or a pharmaceutically acceptable salt, conjugate, hydrate, solvate, polymorph, or mixture thereof, and wherein the bosentan is administered in a single or divided dose of greater than 300 mg/day. In some embodiments, the formulation comprises saquinavir or a pharmaceutically acceptable salt, conjugate, hydrate, solvate, polymorph, or mixture thereof, and wherein the saquinovir is administered in a single or divided dose of greater than 3.8 g/day. In some embodiments, the formulation comprises ritonavir or a pharmaceutically acceptable salt, conjugate, hydrate, solvate, polymorph, or mixture thereof, and wherein the ritonavir is administered in a single or divided dose of greater than 2 g/day. In some embodiments, the formulation comprises efavirenz or a pharmaceutically acceptable salt, conjugate, hydrate, solvate, polymorph, or mixture thereof, and the efavirenz is administered in a single or divided dose of greater than 800 mg/day.

In some embodiments, the formulation comprises at least two of: (i) ketoconazole or a pharmaceutically acceptable salt, conjugate, hydrate, solvate, polymorph, or mixture thereof; (ii) trichloroethylene or a pharmaceutically acceptable salt, conjugate, hydrate, solvate, polymorph, or mixture thereof; (iii) troglitazone or a pharmaceutically acceptable salt, conjugate, hydrate, solvate, polymorph, or mixture thereof; (iv) bosentan or a pharmaceutically acceptable salt, conjugate, hydrate, solvate, polymorph, or mixture thereof; (v) saquinavir or a pharmaceutically acceptable salt, conjugate, hydrate, solvate, polymorph, or mixture thereof; (vi) ritonavir or a pharmaceutically acceptable salt, conjugate, hydrate, solvate, polymorph, or mixture thereof; and (vii) efavirenz or a pharmaceutically acceptable salt, conjugate, hydrate, solvate, polymorph, or mixture thereof.

In some embodiments, the formulation is administered intravenously, intra-arterially, sublingually, transdermally, via an implantable device, subcutaneously, transmucosally, intramuscularly.

Some embodiments provide for the use of at least two of: (i) ketoconazole or a pharmaceutically acceptable salt, conjugate, hydrate, solvate, polymorph, or mixture thereof; (ii) trichloroethylene or a pharmaceutically acceptable salt, conjugate, hydrate, solvate, polymorph, or mixture thereof; (iii) troglitazone or a pharmaceutically acceptable salt, conjugate, hydrate, solvate, polymorph, or mixture thereof; (iv) bosentan or a pharmaceutically acceptable salt, conjugate, hydrate, solvate, polymorph, or mixture thereof; (v) saquinavir or a pharmaceutically acceptable salt, conjugate, hydrate, solvate, polymorph, or mixture thereof; (vi) ritonavir or a pharmaceutically acceptable salt, conjugate, hydrate, solvate, polymorph, or mixture thereof; and (vii) efavirenz or a pharmaceutically acceptable salt, conjugate, hydrate, solvate, polymorph, or mixture thereof for the manufacture of a medicament useful for treating atherosclerotic plaque.

In some embodiments, there is provided a method of treating atherosclerosis in a mammal, comprising administering to a mammal a pharmaceutical formulation comprising a compound, wherein the compound produces a diversion of a bile acid from an enterohepatic circulation to a systemic circulation of the mammal, and wherein the bile acid diverted to the systemic concentration emulsifies an atherosclerotic plaque, resulting in a regression of atherosclerotic plaque.

In some embodiments, the compound comprises at least one of ketoconazole, trichloroethylene, troglitazone, bosentan, saquinavir, ritonavir, and efavirenz. In some embodiments, the diverted bile acid concentration in the systemic circulation ranges from 1 μM to 10 μM, from 10 μM to 50 μM, from 50 μM to 100 μM, from 100 μM to 300 μM ranges from 50 μM to 600 μM.

In some embodiments, the diverted bile acid comprises deoxycholic acid. In some embodiments, the concentration of deoxycholic acid in the systemic circulation is greater than 50 μM. In some embodiments, the concentration of deoxycholic acid in the systemic circulation ranges from 50 μM to 600 μM. In some embodiments, the concentration of deoxycholic acid in the systemic circulation ranges from 100 μM to 300 μM.

In some embodiments, the compound comprises ketoconazole, and wherein the ketoconazole is administered in a single or divided dose that ranges from greater than 50 mg/kg to 166 mg/kg. In some embodiments, the compound comprises trichloroethylene, and wherein the trichloroethylene is administered in a single or divided dose that ranges from greater than 132 mg/kg to 20,000 mg/kg. In some embodiments, the compound comprises troglitazone and the troglitazone is administered in a single or divided dose that ranges from greater than 4.6 mg/kg to 500 mg/kg. In some embodiments, the compound comprises bosentan and the bosentan is administered in a single or divided dose that ranges from greater than 1.8 mg/kg to 500 mg/kg. In some embodiments, the compound comprises saquinavir and the saquinovir is administered in a single or divided dose that ranges from greater than 17.2 mg/kg to 1,000 mg/kg. In some embodiments, the compound comprises ritonavir, and the ritonavir is administered in a single or divided dose that ranges from greater than 8.6 mg/kg to 1000 mg/kg. In some embodiments, the compound comprises efavirenz, and the efavirenz is administered in a single or divided dose that ranges from greater than 8.6 mg/kg to 1000 mg/kg.

In some embodiments, the formulation comprises at least two of ketoconazole, trichloroethylene, troglitazone, bosentan, saquinavir, ritonavir, and efavirenz.

In some embodiments, the formulation is administered intravenously, intra-arterially, orally, sublingually, transdermally, via an implantable device, by injection, or transmucosally.

In some embodiments, a pharmaceutical compound or formulation has the property of diverting at least one endogenous bile salt and/or bile acid from the enterohepatic circulation of a mammal to the systemic circulation in amounts at which the diverted bile salt and/or bile acid is effective to emulsify and dissolve an atherosclerotic plaque, and thereby promote a regression of the size of the atherosclerotic plaque.

In some embodiments, a pharmaceutical compound or formulation \ has the property of diverting at least one endogenous bile salt and/or bile acid from the enterohepatic circulation of a mammal to the systemic circulation in amounts at which the diverted bile salt and/or bile acid is effective to emulsify circulating lipids, and thereby inhibit or prevent atherogenesis.

Some embodiments of the pharmaceutical compounds or formulations comprise, without limitation, ketoconazole, trichloroethylene, troglitazone, bosentan, saquinavir, ritonavir, and efavirenz. Some embodiments comprise these compounds alone, in combination with each other, in combination with other pharmaceutical agents, and in pharmaceutically acceptable formulations.

Some embodiments provide for the use of at least two of ketoconazole, trichloroethylene, troglitazone, bosentan, saquinavir, ritonavir, and efavirenz for the manufacture of a medicament for the treatment of atherosclerotic plaques. In some embodiments, the medicament comprises a dose of ketoconazole that ranges from greater than 50 mg/kg to 166 mg/kg. In some embodiments, the medicament comprises a dose of trichloroethylene that ranges from greater than 132 mg/kg to 20,000 mg/kg. In some embodiments, the medicament comprises a dose of troglitazone that ranges from greater than 4.6 mg/kg to 500 mg/kg. In some embodiments, the medicament comprises a dose of bosentan that ranges from greater than 1.8 mg/kg to 500 mg/kg. In some embodiments, the medicament comprises a dose of saquinavir that ranges from greater than 17.2 mg/kg to 1,000 mg/kg. In some embodiments, the medicament comprises a dose of ritonavir that ranges from greater than 8.6 mg/kg to 1000 mg/kg. In some embodiments, the medicament comprises a dose of efavirenz that ranges from greater than 8.6 mg/kg to 1000 mg/kg. In some embodiments, the medicament comprises at least two of ketoconazole, trichloroethylene, troglitazone, bosentan, saquinavir, ritonavir, and efavirenz.

### DETAILED DESCRIPTION OF THE INVENTION

One approach in the treatment of atherosclerosis has been to use pharmacologic agents to interfere with the synthesis of cholesterol, a component of LDL, a major component of the lipid core of the plaque. It is oxidized LDL that provides, at least in part, the primary insult to the vessel wall that results in infiltration of monocytes, their differentiation into macrophages, and the inflammatory reactions that ensues. For example, statins are now a drug of choice in the treatment of atherosclerosis on the basis of their ability to decrease cholesterol synthesis by interfering with the enzyme HMG-CoA reductase.

Other approaches have devised ways in which to stabilize plaques, so that the risk of rupture and the attendant possibility of an acute coronary event is minimized or removed. Other approaches include treating plaque locally with antithrombolytics in order to prevent the complications due to clot formation after plaque rupture, for example as disclosed in International Patent Application No. PCT/IN2006/000037 (Chandrasekar).

Despite the relatively widespread use of statins to treat atherosclerosis, at the normally prescribed doses these compounds only reduce but do not eliminate the risk of acute coronary events due to atherosclerotic plaque. As a result, there remains a need to a way in which to reduce plaque volume in patients, in essence to reverse the progression of atherosclerosis, by causing the regression of existing plaques.

U.S. Pat. No. 7,141,045 (Johansson et al.) discloses a method of dissolving plaque by direct application of a dissolution fluid through an intravascular catheter. The dissolution fluid can include a variety of detergents, surfactants, and other solubilizing agents, in addition to enzymes, and metal ion chelators. While such an approach might be useful for acute treatment of known atherosclerotic lesions, it is seriously limited in it utility. First, the procedure is invasive, such that it can only be performed by a surgeon in an operating room situation. This necessarily means the procedure will be costly. Second, the treatment is only effective for plaques that can be effectively reached by catheter, and only for plaques whose location is known well enough by imaging techniques, such that the catheter can be guided to the desired location. Local treatment is thus generally ineffective as a sole method for the systemic treatment of atherosclerotic plaque.

As a result, there remains a need for noninvasive, systemically effective compositions and treatments that we effective to result in solubilization and regression of atherosclerotic plaque, especially soft, or vulnerable, plaque. Results from prior studies, testing whether statins were effective to cause plaque regression, have been described as equivocal. For example, in the recently completed ASTEROID study (Nissen et al., (2006), JAMA 295: 1556-1565), experiments were designed to test whether 40 mg/day of rosuvastatin would be effective to result in a decrease in plaque volume, as evidenced by intravascular ultrasound imaging techniques. While the treatment was particularly effective at modulating LDL, HDL, and triglyceride levels, plaque volume after 2 years was only reduced by 8.5% (SD=13.7) in the most diseased segments of vessels examined, and by only 6.7% (SD=11.1) with respect to normalized total atheroma volume. Thus, statins are not particularly effective at producing significant reductions in plaque burden, even when provided at twice the normally prescribed dosage for a period of two years.

Some embodiments use emulsifiers provided either systemically or locally to dissolve plaque and result in plaque regression. Emulsifiers can include bile salts, saponins, and various detergents.

Bile acids are cholesterol-derived organic acids that have detergent properties. Bile acids play important roles physiologically in the absorption, transport, and secretion of lipids. These compounds have been characterized as primary or secondary bile acids, depending on whether they are synthesized de novo (primary) or are derived by subsequent chemical modification (secondary). Primary bile acids are produced by the liver and include cholic acid (3α, 7α, 12α,-trihydroxy-5β-cholanic acid) and chenodeoxycholic acid (3α, 7α,-dihydroxy-β-cholanic acid). Dehydroxylation of the primary bile acids, for example by intestinal bacteria, produces the more hydrophobic secondary bile acids, for example deoxycholic acid (3α, 12α,-dihydroxy-5β-cholanic acid), and lithocholic acid (3α-hydroxy-5β-cholanic acid). Together, the primary and secondary bile acids make up about 99% of the total bile acid pool in humans.

The role of circulating bile acid levels in the development of atherosclerosis is not clear in the prior art. Previous studies in animal model systems have suggested that lowering circulating levels of bile acids through the use of bile acid sequestrants lowers LDL levels and results in regression of atherosclerotic plaque (Wissler, J. Clin. Apher. 4: 52-58, 2006). The bile acid sequestrants colesevelam HCl has been shown to reduce LDL particle number and increase LDL particle size in patients with hypercholesterolemia (Rosenson, Atheroscl. 185: 327-330, 2006). Dietary supplements comprising bile acid polymeric organic bases have been shown to inhibit cholesterol rise and atherosclerotic plaque formation in chickens on a high cholesterol diet (Tennent et al., J. Lip. Res. 1: 469-473, 1960). Thus, collectively the prior art suggests that decreasing circulating bile acid levels should be effective to reduce progression, or even promote regression of atherosclerotic plaques.

Contrary to these prior art studies, where reducing circulating levels of bile salts is predicted to slow or regress plaque, some embodiments of the present disclosure teach formulations and methods that lead to a sustained increase in the level of emulsifiers in the systemic circulation. These levels are effective to dissolve the lipid components of atherosclerotic plaque, especially vulnerable plaque, leading to plaque regression. In some embodiments, the emulsifiers comprise bile acids. In some embodiments, the emulsifiers are detergents, for example, ionic detergents, nonionic detergents, and zwitterionic detergents. In some embodiments, the emulsifiers comprises saponins. In some embodiments, the emulsifiers comprise combinations of bile acids, detergents, and/or saponins. Experimental examples described below demonstrate that bile salt emulsifiers can be effective to dissolve the lips core of atherosclerotic plaque.

There are instances where the concentration of bile acids have been increased systemically. For example, it has been previously shown that feeding hyodeoxycholic acid (HDCA) to C57BL/6 LDL r-KO knockout mice (genetically predisposed to develop atherosclerosis) results in a reduced rate of formation of atherosclerotic plaque relative to mice not provided HDCA (Sehayek et al., J. Lip. Res. 42: 1250-1256, 2001). Plasma levels of wild type mice, provided the same amount of dietary HDCA, ranged up to about 50 μM. However, there is no evidence that these levels were effective to result in plaque regression, as is provided by some embodiments described herein.

Primary biliary cirrhosis (PBC) is an inflammatory disease characterized by destruction of the small bile ducts within the liver, eventually leading to cirrhosis. While the cause of PBC is not precisely known, the presence of autoantibodies in PBC patients suggests an autoimmune origin. Among the various symptoms that arise as a result of PBC, it is known that total plasma cholesterol tends to be elevated, by as much as 50%. Despite the increases in cholesterol levels, however, it appears that PBC patients are not at an increased risk of atherosclerosis. In addition, it has been shown that PBC patients have elevated levels of bile acids (Murphy et al., Gut 13: 201-206, 1972), with levels averaging about 200 μM, as compared to normal levels which are less than 10 μM. Thus, some embodiments as described herein are effective to mimic the high levels of bile salts observed in PBC patients, and in doing so are effective to result in regression of atherosclerotic plaque.

Some embodiments comprise pharmaceutical compounds or formulations useful in the treatment of atherosclerotic plaques. Some embodiments comprise pharmaceutical compounds or formulations that have the property of producing a diversion, from the enterohepatic circulation of a mammal to the systemic circulation, of endogenous bile acids, bile salts, their precursors, and their derivatives in concentrations effective to emulsify and dissolve atherosclerotic plaques and plaque components, especially lipids such as cholesterol, either in a plaque or in circulation. This property may result in a size regression of an atherosclerotic plaque, an inhibition of atherosclerotic plaque formation, a restoration of patency to an arterial vessel obstructed by an atherosclerotic plaque, and combinations thereof. This property may also result in an inhibition of long term hypoxic tissue damage associated with reduced blood flow from arterial occlusion, such as cardiomyopathy, heart failure, senile dementia, vascular complications from diabetes, nephrosclerosis, systemic and pulmonary hypertension, mesenteric ischemia, cerebral atherosclerosis, macular degeneration, and Alzheimer disease, likely a result of anoxic chronic insults of various etiology all converging into inadequate cerebral perfusion mainly to the cognition and memory centers.

As used herein, the term “mammal” includes humans and human patients in need of atherosclerotic plaque treatment.

In some embodiments, a pharmaceutical compound or formulation is effective to inhibit complications of atherosclerosis, such as acute coronary events, thrombus formation, and cerebrovascular accidents. In some embodiments, a pharmaceutical compound or formulation is useful for inhibiting peripheral vascular disease, such as ischemic limb disease, and complications associated with vascular disease, such as amputation.

### Examples of Bile Acid Diverting Compounds

In some embodiments, the dissolving/emulsifying action exerted upon preexisting atherosclerotic plaques as well as the beneficial effects on atherogenesis produced by the pharmaceutical compounds and formulations are achieved by making endogenous bile acids bioavailable in the systemic circulation of a mammal in pharmacologically active concentrations. An increase of endogenous bile acids and/or bile salts in the systemic circulation to pharmacologically active concentrations can be accomplished by diverting them from the enterohepatic circulation to the systemic circulation.

In some embodiments of the present invention, bile salts and/or acids may be diverted from the enterohepatic circulation of a mammal to the systemic circulation by inhibiting bile acid and/or bile salt uptake receptors located in hepatocyte cell walls. In some embodiments of the present invention, bile salts and/or acids may be diverted from the enterohepatic circulation of a mammal to the systemic circulation by inhibiting their intracellular transport from the sinusoidal pole to the bile pole of a hepatocyte. In some embodiments of the present invention, bile salts and/or acids may be diverted from the enterohepatic circulation of a mammal to the systemic circulation by inhibiting their excretion from a hepatocyte. In some embodiments of the present invention, bile salts and/or acids may be diverted from the enterohepatic circulation of a mammal to the systemic circulation by simultaneously inhibiting two or more of the uptake, the intracellular transport, and the excretion of bile salts and/or bile acids by hepatocytes.

Some embodiments of the pharmaceutical compounds and/or formulations comprise, without limitation, ketoconazole, trichloroethylene, troglitazone, bosentan, saquinavir, ritonavir, and efavirenz. Some embodiments comprise these compounds alone, in combination with each other, in combination with other pharmaceutical agents, and in pharmacologically acceptable formulations.

Prior studies have demonstrated that 25 mg/kg ketoconazole produces an increase in serum levels of cholic acid, taurocholic acid, and chenodeoxycholic acid; whereas 50 mg/kg of ketoconazole produces an increase in the serum levels of cholic acid, taurocholic acid, chenodeoxycholic acid, glycocholic acid, glycochenodeoxycholic acid, glycodeoxycholic acid, deoxycholic acid and taurochenodeoxycholic acid by strongly inhibiting their hepatocellular uptake in a relatively specific fashion. These inhibitory effects of ketoconazole on bile acid uptake involves a dose related pharmaceutical effect of ketoconazole. (Azer et al., (1995) Journal of Pharmacology and Experimental Therapeutics. 272(3):1231-1237, the entire contents of which are hereby incorporated by reference in their entirety). But prior studies have also reported hepatotoxicity as a side effect of ketoconazole. Still, the mechanism by which ketoconazole achieves inhibition of bile acid uptake by hepatocytes appears distinct from the mechanism that gives rise to hepatotoxicity. (Azer et al. (1995)).

In addition, previous studies have demonstrated that 1 mmol/kg trichloroethylene causes serum accumulation of bile acids by producing a reversible physiological interference to bile acid uptake by hepatocyte receptors, rather than causing an event associated with significant pathological consequences such as actual cell damage. (Bai et al. (1993) Toxicology and Applied Pharmacology. 121(2):296-302, the entire contents of which are hereby incorporated by reference in their entirety).

Accordingly, stereochemical configuration of ketoconazole and trichloroethylene may be used to elucidate the molecular geometry of bile acids binding sites of the bile acid uptake receptors present on hepatocyte membranes, and such information may be used to identify and/or design low toxicity compounds that competitively bind to the bile acid binding sites of hepatocyte bile acid receptors with high affinity. Such compounds would have the properties of lacking detrimental side effects, reversibly inhibiting bile acids uptake by hepatocytes, and inducing a significant increase in serum concentrations of endogenous bile acids.

Previous studies have also demonstrated that 10 μM troglitazone and 100 μM bosentan inhibit both hepatic uptake and excretion of bile acids from the bile pole of the hepatocyte in sandwich culture rat hepatocyte systems, and may therefore divert bile acids from the enterohepatic circulation to the systemic circulation. (Kemp et al. (2004) Toxilogical Science. 83(2):207-214, the entire contents of which are hereby incorporated by reference in their entirety). In addition, prior studies have demonstrated that, 28 μM ritonavir, 15 μM saquinavir, and 32 μM efavirenz, but not nevirapine, inhibit bile acid transport in sandwich culture human and rat hepatocytes, and may therefore divert bile acids from the enterohepatic circulation to the systemic circulation. (McRae et al. (2006). The Journal of Pharmacology and Experimental Therapeutics. 318(3):1068-1075, the entire contents of which are hereby incorporated by reference in their entirety). bile

Although prior studies have indicated that ketoconazole, trichloroethylene, troglitazone, bosentan, saquinavir, ritonavir, and efavirenz produce a diversion of endogenous bile acids from the enterohepatic to the systemic circulation of a mammal, no prior art study has indicated doses and/or combinations of those compounds effective to produce an increase the concentration of bile salts and/or acids in the systemic circulation to levels at which the diverted bile acids and/or bile salts emulsify and dissolve atherosclerotic plaques, resulting in a regression of plaque size. Nor has any prior art study indicated doses and/or combinations of those compounds effective to produce an increase in the concentration of bile salts and/or acids in the systemic circulation to levels at which they emulsify free circulating lipid components of atherosclerotic plaques, such as cholesterol. Some embodiments comprise such bile acid and/or bile salt diverting doses and combinations.

### Examples of Doses of Bile Acid Diverting Compounds

Some embodiments of effective doses of bile acid diverting compounds of the present invention, when administered alone, in combination, and/or in formulations, comprise amounts sufficient to produce a diversion of at least one endogenous bile acid, bile salt, bile acid precursor, bile salt precursor, bile acid derivative, and bile salt derivative from the enterohepatic circulation of a mammal to the systemic circulation such that the bile acid, bile salt, bile acid precursor, bile salt precursor, bile acid derivative, and/or bile salt derivative achieve systemic circulation concentrations effective to emulsify and dissolve atherosclerotic plaques and plaque components, thereby resulting in an atherolytic or antiatherogenic effect. Some embodiments comprising ketoconazole as the single active bile acid diverting compound comprise ketoconazole doses above 600 mg/day. Some embodiments comprising ketoconazole as the single active bile acid diverting compound comprise ketoconazole doses above 600 mg/day for at least seven days. Some embodiments comprising trichloroethylene as the single active bile acid diverting compound comprise trichloroethylene doses above 135 mg/kg. Some embodiments comprising trichloroethylene as the single active bile acid diverting compound comprise trichloroethylene doses above 135 mg/kg/day for at least seven days. Some embodiments comprising troglitazone as the single active bile diverting compound comprise troglitazone doses above 30 mg/kg/day. Some embodiments comprising troglitazone as the single active bile diverting compound comprise troglitazone doses above 500 mg/day for at least 28 days. Some embodiments comprising bosentan as the single active bile diverting compound comprise bosentan concentrations above 300 mg/day. Some embodiments comprising saquinavir as the single active bile diverting compound comprise saquinavir doses above 3.8 g/day. Some embodiments comprising ritonavir as the single active bile diverting compound comprise ritonavir doses above 2 g/day. Some embodiments comprising efavirenz as the single active bile diverting compound comprise efavirenz doses above 800 mg/kg.

Some embodiments of ketoconazole doses include 1 to 25 mg, 25 to 50 mg, 50 to 75 mg, 75 to 100 mg, 100 to 150 mg, 150 to 200 mg, 200 to 250 mg, 250 to 300 mg, 300 to 400 mg, 400 to 500 mg, 500 to 600 mg, 600 to 700 mg, 700 to 800 mg, 800 to 900 mg, 900 to 1000 mg, 1000 to 1250 mg, 1250 to 1500 mg, 1500 to 2000 mg, and greater than 600 mg.

Some embodiments of trichloroethylene doses include 1 to 1,000 mg/kg, 1,000 to 2,000 mg/kg, 2,000 to 3,000 mg/kg, 3,000 to 4,000 mg/kg, 4,000 to 5000 mg/kg, 5,000 to 6,000 mg/kg, 6,000 to 7,000 mg/kg, 7,000 to 8,000 mg/kg, 8,000 to 9,000 mg/kg, 10,000 to 11,000 mg/kg, 11,000 to 12,000 mg/kg, 12,000 to 13,000 mg/kg, 13,000 to 14,000 mg/kg, 14,000 to 15,000 mg/kg, 15,000 to 16,000 mg/kg, 16,000 to 17000 mg/kg, 17,000 to 18,000 mg/kg, 18,000 to 19,000 mg/kg, 19,000 to 20,000 mg/kg, 1 to 20,000, 1,000 to 19,000 mg/kg, 2,500 to 17,500 mg/kg, 5,000 to 15,000 mg/kg, 7,500 to 12,500 mg/kg, 10,000 to 11,000 mg/kg and greater than 135 mg/kg.

Some embodiments of troglitazone doses include 1 to 5 mg/kg, 5 to 10 mg/kg, 10 to 20 mg/kg, 20 to 30 mg/kg, 30 to 40 mg/kg, 40 to 50 mg/kg, 50 to 75 mg/kg, 75 to 100 mg/kg, 100 to 125 mg/kg, 125 to 150 mg/kg, 150 to 200 mg/kg, 200 to 250 mg/kg, 250 to 300 mg/kg, 300 to 400 mg/kg, 400 to 500 mg/kg, and greater than 4.6 mg/kg to 500 mg/kg

Some embodiments of bosentan doses include 1 to 10 mg/day, 10 to 50 mg/day, 50 to 100 mg/day, 100 to 150 mg/day, 150 to 200 mg/day, 200 to 300 mg/day, 400 to 500 mg/day, 500 to 600 mg/day, 600 to 750 mg/day, 750 to 1000 mg/day, 1000 to 1250 mg/day, 1250 to 1500 mg/day, 1500 to 1750 mg/day, 1750 to 2000 mg/day, and greater than 300 mg/day.

Some embodiments of saquinavir doses include 1 to 100 mg/day, 100 to 200 mg/day, 200 to 250 mg/day, 250 to 300 mg/day, 300 to 400 mg/day, 400 to 500 mg/day, 500 to 600 mg/day, 600 to 700 mg/day, 700 to 800 mg/day, 800 to 900 mg/day, 900 to 1,000 mg/day, 1 to 5 g/day, 5 to 10 g/day, and greater than 3.8 g/day.

Some embodiments of ritonavir doses include 1 to 100 mg/day, 100 to 200 mg/day, 300 to 400 mg/day, 400 to 500 mg/day, 500 to 600 mg/day, 600 to 700 mg/day, 700 to 800 mg/day, 800 to 900 mg/day, 900 to 1,000 mg/day, 1 to 5 g/day, 5 to 10 g/day, and greater than 2 g/day.

Some embodiments of efavirenz doses include 1 to 100 mg/day, 100 to 200 mg/day, 200 to 250 mg/day, 250 to 300 mg/day, 300 to 400 mg/day, 400 to 500 mg/day, 500 to 600 mg/day, 600 to 700 mg/day, 700 to 800 mg/day, 800 to 900 mg/day, 900 to 1,000 mg/day, 1 to 5 g/day, 5 to 10 g/day, and greater than 800 mg/day.

### Examples of Bile Acids

As used herein, the terms “bile acid” and “bile salt” each include bile acids, bile salts, precursors of bile acids, precursors of bile salts, derivative of bile acids, and derivatives of bile salts. Bile acids and bile and bile salts, as used herein, can include cholic acid, chenodeoxycholic acid, deoxycholic acid, lithocholic acid, ursodeoxycholic acid, hyodeoxycholic acid, taurocholic acid, glycocholic acid, glycochenodeoxycholic acid, glycodeoxycholic acid, and taurochenodeoxycholic acid.

Bile acids useful in some embodiments can include, without limitation: 1,3,12-trihydroxycholanoic acid; 1,3,7,12-tetrahydroxycholanoic acid; 3beta-hydroxy-delta 5-cholenic acid; 3 beta-hydroxychol-3-en-24-oic acid; 3′-isøthiocyanatobenzamidecholic acid; 3,12-dihydroxy-5-cholenoic acid; 3,4,7-trihydroxycholanoic acid; 3,6,12-trihydroxycholanoic acid; 3,7,12,23-tetrahydroxycholan-24-oic acid; 3,7,12-trihydroxy-7-methylcholanoic acid; 3,7,12-trihydroxycoprostanic acid; 3,7,23-trihydroxycholan-24-oic acid; 3,7-dihydroxy-22,23-methylene-cholan-24-oic acid (2-sulfoethyl)amide; 3-((3-cholamidopropyl)dimethylammonium)-1-propanesulfonate; 3-((3-deoxycholamidopropyl)dimethylammonio)-1-propane; 3-benzoylcholic acid; 3-hydroxy-5-cholen-24-oic acid 3-sulfate ester; 3-hydroxy-7-(hydroxyimino)cholanic acid; 3-\ddot{ι}odocholic acid; 7,12-dihydroxy-3-(2-(glucopyranosyl)acetyl)cholan-24-oic acid; 7,12-dihydroxy-3-oxocholanic acid; allocholic acid; chapso; chol-3-en-24-oic acid; cholanic acid; sodium cholate; methyl cholate; benzyldimethylhexadecylammonium cholate; methyl 1,3-dihydroxycholan-24-oate; and trioctylmethylammonium cholate); cholic acid glucuronide; cholyl-coenzyme A; cholyl-lysylfluorescein; cholyldiglycylhistamine; cholylhistamine; cholylhydroxamic acid; cholylsarcosine; cholyltetraglycylhistamine; ciliatocholic acid; dehydrocholic ccid (which includes FZ 560; Gallo-Merz; Gillazym; Hepavis; Mexase; progresin Retard; and spasmocanulase); 23-nordeoxycholic acid; 3,7-dioxocholanoic acid; 3-hydroxy-ρolydeoxycholic acid; 3-sulfodeoxycholic acid; 6-hydroxycholanoic acid; 6-methylmurideoxycholic acid; 7-ketodeoxycholic acid; 7-methyldeoxycholic acid; chenodeoxycholic acid; dehydrodeoxycholic acid; deoxycholyltyrosine; desoxybilianic acid; glycodeoxycholic acid; hyodeoxycholate-6-O-glucuronide; hyodeoxycholic acid; taurodeoxycholic Acid; and ursodeoxycholic acid; glycocholic acid; 3-hydroxy-5-cholenoylglycine; cholylglycylhistamine; cholylglycyltyrosine; glycodeoxycholic Acid; sulfolithocholylglycine; hemulcholic acid; 12-ketolithocholic acid; 24-norlithocholic acid; 3-dehydrolithocholylglycine; 3-hydroxy-6-cholen-24-oic acid; 3-hydroxy-7,12-diketocholanoic acid; 3-hydroxy-7-methylcholanoic acid; 3-ketolithocholic acid; 3-oxochol-4-en-24-oic acid; 3-oxocholan-24-oic acid; 4-azidophenacyl lithocholate; 7-ketolithocholic acid; BRL 39924A; glycolithocholic acid; lithocholate 3-O-glucuronide; lithocholyl-N-hydroxysuccinimide; methyl lithocholate; N-carbobenzoxy-N-lithocholyl-epsilon-lysine; N-epsilon-lithochoiyllysine; sulfolithocholic acid; and taurolithocholic acid; muricholic acid; N-(1,3,7,12-tetrahydroxycholan-24-oyl)-2-aminopropionic acid; N-(2-aminoethyl)-3,7,12-trihydroxycholan-24-amide; N-carboxymethyl)-N-(2-(bis(carboxymethyl)amino)ethyl)-3-(4-(N′-(2-((3,7,12-trihydroxycholan-24-oyl)araino)ethyl)(thioureido)ρhenyl)alanine; N-cholyl-2-fluoro-beta-alanine; norcholic acid; norursocholic acid; taurocholic acid; (N-(7-(nitrobenz-2-oxa-1,3-diazol-4-yl))-7-amino-3alpha, 12alpha-dihydroxycholan-24-oyl)-2-aminoethanesulfonate; 23-seleno-25-homotaurocholic acid; 3,12-dihydroxy˜7˜oxocholanoyltaurine; 3-hydroxy-7-oxocholanoyltaurine; azidobenzamidotaurocholate; hexadecyltributylammonium taurocholate; tauro 1-hydroxycholic acid; tauro-3,7-dihydroxy-12-ketocholanoic acid; taurodehydrocholate; taurodeoxycholic acid; tauroglycocholic acid; taurolithocholic acid; tauromurichoUc acid; tauronorcholic acid); tetrahydroxy-5-cholan-24-oic acid; ursocholic acid; vulpecholic acid; bile acid sulfates; glycodeoxycholic acid; glycochenodeoxycholic acid; 7-oxoglycochenodeoxycholic acid; glycochenodeoxycholate-3-sulfate; glycohyodeoxycholic acid; tauro-7,12-dihydroxycholanic acid; taurochenodeoxycholic acid; taurochenodeoxycholate-3-sulfate; taurochenodeoxycholate-7-sulfate; tauroursodeoxycholic acid; taurohyodeoxycholic acid; the includes: 23-methylursodeoxycholic acid; 24-norursodeoxycholic acid; 3,6-dihj^{?}droxy-6-methylcholanoic acid; 3,7-dihydroxy-20,22-methylenecholan-23-oic acid; 3,7-dihydroxy-22,23-methylenecholan-24-oic acid; 3,7-dihydroxy-7-ethylcholanoic acid; 3,7-dihydroxy-7-methylcholanoic acid; 3,7-dihydroxy-7-n-propylcholanoic acid; Bamet-UD2; diammhiebis(ursodeoxycholate(O,O′))platinum(II); glycoursodeoxycholic acid; homoursodeoxycholic acid; HS1030; HS1183; isoursodeoxycholic acid; PABA-ursodeoxycholic acid; sarcosylsarcoursodeoxycholic acid; sarcoursodeoxycholic acid; ursodeoxycholate-3-sulfate; ursodeoxycholic acid 7-oleyl ester; ursodeoxycholic acid N-acetylglucosaminide; ursodeoxycholic acid-3-O-glucuronide; ursodeoxycholyl N-carboxymethylglycine; ursodeoxycholylcysteic acid; ursometh; 24-norchenodeoxycholic acid; 3,7-dihydroxy-12-oxocholanoic acid; 3,7-dihydroxy-24-norcholane-23-sulfonate; 3,7-dihydroxy-25-homocholane-25-sulfonate; 3,7-dihydroxychol-5-enoic acid; 3,7-dihydroxycholane-24-sulfonate; 3-glucosido-chenodeoxycholic acid; 3-oxo-7-hydroxychol-4-enoic acid; 6-ethylchenodeoxycholic acid; chenodeoxycholate sulfate conjugate; chenodeoxycholyltyrosine; glycochenodeoxycholic acid which includes: 7-oxoglycochenodeoxycholic acid and glycochenodeoxycholate-3-sulfate; homochenodeoxycholic acid; HS 1200; methyl 3,7-dihydroxychol-4-en-24-oate; methyl 3,7-dihydroxycholanate; N-(2-aminoethyl)-3,7-dihydroxycholan-24-amide; N-chenodeoxycholyl-2-fluoro-beta-alanine; sarcochenodeoxycholic acid; taurochenodeoxycholic acid; taurochenodeoxycholate-3-sulfate; taurochenodeoxycholate-7-sulfate; tauroursodeoxycholic acid.

### Examples of Systemic Circulation Concentrations of Bile Acids

Some embodiments of systemic circulation concentrations of a bile acid and/or a bile salt effective to result in regression of atherosclerotic plaque may vary depending on a number of factors. Influential variables can include, for example, various chemical properties of one bile acid and/or a bile salt as compared to another. For example different bile acids and/or a bile salts can have differing p_{Ka} values or solubility, and these properties of a particular bile acid may affect how a patient metabolizes the bile acid, how much of the bile acid may remain in the circulation, and how effective the bile acid may be in emulsifying and dissolving atherosclerotic plaques.

Accordingly, in some embodiments of the present invention, a systemic circulation concentration of a bile acid and/or a bile salt effective to emulsify and dissolve atherosclerotic plaques ranges from 1 μM to 10 μM, 10 μM to 50 μM, 5 μM to 10 μM, 10 μM to 20 μM, 20 μM to 30 μM, 30 μM to 40 μM, 40 μM to 50 μM, 50 μM to 60 μM, 60 μM to 70 μM, 70 μM to 80 μM, 80 μM to 90 μM, 90 μM to 100 μM, 50 μM to 600 μM, 50 μM to 100 μM, 100 μM to 300 μM, 100 μM to 550 μM, 150 μM to 500 μM, 200 μM to 450 μM, 250 μM to 400 μM, and 300 μM to 350 μM.

### Examples of Saponin Emulsifiers

In some embodiments, saponins are provided as emulsifiers. Saponins are naturally occurring compounds predominantly derived from plants and which have detergent properties. The name saponin is derived from the soapwort plant (Saponaria) traditional used in the making of a type of soap. Saponins are the glycosides of 27 carbon steroids or 30 carbon triterpenes. Removal of the sugar moiety from a saponin by hydrolysis yields the aglycone, sapogenin. Triterpenoid saponins are generally acid, while steroid saponins are generally neutral.

Steroid saponins include three classes of compounds, the cholestanol, furostanol, and spirostanol saponins. Examples of furostanol saponins can include, proto-isoeruboside-B and isoeruboside-B, as well as saponins derived, for example, from Ruscus aculeatus, Tacca chantrieri, Solanum hispidum, Dioscorea polygonoides, Tribulus terrestris, and Lilium candidum. Other steroid saponins can include those derived from Saponaria officinalis, Yucca schidigera, and Chlorogalum pomeridianum.

Examples of triterpenoid saponins can include those of the fusidane-lanostante group, cyclopassiflosides, cycloglobiseposides, cycloartanes, dammaranes (e.g., bacopasaponin and jujubogenin), lupanes (e.g., quadranosides), oleananes (e.g., maesapinin), ligatosides, sandrosaponins, pedunsaponins), vulgarsaponin, peduncularisaponin, petersaponin, araliasaponin, assamsaponin, eupteleasaponin, herniariasaponin, jeosaponin, meliltussaponin, ursanes (e.g., randisaponins), brevicuspisaponin, ursolic acid, and indicasaponin. Triterpenoids can also be derived from Quillaja saponaria, as well as those derived from grapes.

Saponins have been identified in plants and animals including, for example, and without being limiting, agave, alfalfa, aloe, Anadenanthera peregrine, amaranth, Angelica sinesis, Aralia chinesis, Aralia manshurica, asparagus, Astragalus membranaceus, Bacopa monnieri, Boussingaultia sp., Bupleurum chinense, Calendula officinalis, Capsicum sp., chickweed, Chlorophytum sp., Chlorogalum sp., Codonopsis pilosula, horse chestnuts, curcurbit, Digitalis sp., Echinodermata, Elecampane, Elutherococcus senticosus, fenugreek, goldenrod, gotu kola, grape skin, Gymnema sylvestre, Gypsophila sp., hawthorn, jiaogulan, licorice, lungwort, mullein, olives, onion, pannax (Koren Ginseng), Platycodon grandiflorum, Polygala tenuifola, Quillaja saponaria, quinoa, Phytolacca americana, rambutan, Salvia sp., soapberry, Saponaria sp., Schizandra chinensis, shallots, southern pea, soybean, Tribulus terrestris, wild yam, yucca, and Zizyphus jujube.

### Examples of Saponin Emulsifiers

Various detergents are useful as emulsifiers in some embodiments as described herein, including ionic detergents, nonionic detergents, and zwitterionic detergents. Detergents can be used to augment or enhance the effectiveness of other emulsifiers such as bile acids and/or saponins. Detergent can also be used as permeability enhancers, effective to enhance the permeability of membranes or tissue to emulsifiers.

### Examples of Routes of Administration

Various routes of administration of emulsifiers can be used, for example, and without being limiting, by injection, transdermally, orally, by inhalation, and transmucosally. In some embodiments, emulsifiers can be perfused directly into the systemic circulation by way of an implantable pump. Regardless of the route of administration, the dosing of emulsifiers will result in achieving sustained levels of an emulsifier in the systemic circulation that are effective to result in plaque regression.

In some embodiments, formulations comprise a sustained release formulation that results in the maintenance of circulating levels of emulsifiers that are effective to result in plaque regression. In some embodiments, formulations can comprise a sustained release delivery system can be used to deliver the emulsifier such that increased levels are achieved for extended periods of time, for example, a period of 2 hours or longer. In some embodiments, release is sustained over a period of 24 hours. In some embodiments, a sustained release delivery system can further comprise one or more pharmaceutical diluents known in the art. Exemplary pharmaceutical diluents include, without limitation, monosaccharides, disaccharides, polyhydric alcohols and a combination thereof. In some embodiments, pharmaceutical diluents can include, for example, starch, lactose, dextrose, mannitol, sucrose, microcrystalline cellulose, sorbitol, xylitol, fructose, a combination thereof.

In some embodiments, the pharmaceutical diluent can be water soluble, for example, lactose, dextrose, mannitol, sucrose, and a combination thereof. In some embodiments, the sustained release delivery system can comprise one or more pharmaceutical diluents in an amount of about 5% to about 80% by weight; from about 10% to about 50% by weight; or about 20% by weight of a dosage form.

In some embodiments, a emulsifier delivery system can comprise one or more hydrophobic polymers. The hydrophobic polymers can be used in an amount sufficient to slow the hydration of the active ingredients. For example, the hydrophobic polymer can be present in the sustained release delivery system in an amount of about 0.5% to about 20% by weight; in an amount of about 2% to about 10% by weight; in an amount of about 3% to about 7% by weight; or in an amount of about 5% by weight.

Some embodiments of formulations as described herein can be admixed with one or more wetting agents (e.g., polyethoxylated castor oil, polyethoxylated hydrogenated castor oil, polyethoxylated fatty acid from castor oil, polyethoxylated fatty acid from hydrogenated castor oil, or a combination thereof) one or more lubricants (e.g., magnesium stearate, sodium stearyl fumarate), one or more glidants (e.g., silicon dioxide), one or more buffering agents, one or more colorants, and/or other conventional ingredients well known to those of skill in the art of pharmaceutical compounding.

In some embodiments, a sustained release coating can comprise at least one water insoluble compound, for example, a hydrophobic polymer. The hydrophobic polymer can be the same as or different from the hydrophobic polymer used in the sustained release delivery system. Exemplary hydrophobic polymers include, without being limiting, alkyl celluloses (e.g., C₁₋₆ alkyl celluloses, carboxymethylcellulose), other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes (alone or in admixture with fatty alcohols), shellac, hydrogenated vegetable oils, and a combination thereof. In some embodiments, the hydrophobic polymer can comprise methyl cellulose, ethyl cellulose, propyl cellulose or a mixture of two or more thereof. In another embodiment, the hydrophobic polymer is ethyl cellulose. The compositions of the invention can be coated with a water insoluble compound to a weight gain from about 1 to about 20% by weight.

Formulation can be coated with a sustained release coating that can further comprise at least one plasticizer such as triethyl citrate, dibutyl phthalate, propylene glycol, polyethylene glycol, or mixtures of two or more thereof. A sustained release coating can also contain at least one water soluble compound, such as polyvinylpyrrolidones, hydroxypropylmethylcelluloses, and mixtures thereof.

A sustained release coating can be applied to a core comprising one or more emulsifiers by spraying an aqueous dispersion of the water insoluble compound onto core. The core can be a granulated composition made, for example, by dry or wet granulation of mixed powders of emulsifiers and at least one binding agent; by coating an inert bead with emulsifiers and at least one binding agent; or by spheronizing mixed powders of emulsifiers and at least one spheronizing agent. Some exemplary binding agents include hydroxypropylmethylcelluloses. Exemplary spheronizing agents can include microcrystalline celluloses. The inner core can be a tablet made by compressing the granules or by compressing a powder comprising emulsifiers and/or pharmacologically acceptable salts or conjugates thereof.

In some embodiments, the compositions comprising emulsifiers and a sustained release delivery system, as described herein, are coated with a sustained release coating, as described herein. In some embodiments, the compositions comprising emulsifiers and a sustained release delivery system, as described herein, are coated with a hydrophobic polymer, as described herein. In some embodiments, the compositions comprising emulsifiers and a sustained release delivery system, as described herein, are coated with an enteric coating. Exemplary enteric coatings include, without being limiting, cellulose acetate phthalate, hydroxypropylmethylcellulose phthalate, polyvinylacetate phthalate, methacrylic acid copolymer, shellac, hydroxypropylmethylcellulose succinate, cellulose acetate trimelliate, and a combination thereof.

In some embodiments, the compositions comprising an emulsifier and a sustained release delivery system, as described herein, are coated with a hydrophobic polymer, as described herein, and further coated with an enteric coating. In some embodiments described herein, the compositions comprising emulsifiers and a sustained release delivery system, as described herein, can optionally be coated with a hydrophilic coating which can be applied above or beneath a sustained release film, above or beneath the hydrophobic coating, and/or above or beneath the enteric coating. Exemplary hydrophilic coatings include hydroxypropylmethylcelluloses.

Formulations can further comprise agents to enhance absorption across the intestinal epithelium. These can include, without being limiting, other emulsifiers or detergents, some of which are listed above, EDTA, sodium salicylate, sodium caprate, diethyl maleat, N-lauryl-β-D-maltophyranoside, linoleic acid polyoxyethylated, tartaric acid, SDS, Triton X-100, hexylglucoside, hexylmaltoside, heptylglucoside, octylglucoside, octylmaltoside, nonylglucoside, nonylmaltoside, decylglucoside, deceylmaltoside, dodecylmaltoside, tetradecylmaltoside, dodecylglucoside, tridecylmaltoside, as well as mucolytic agents, for example N-acetylcysteine and chitosan.

Where a transdermal route is selected, the formulation can further comprise one or more permeability enhancers, effective to increase the rate of movement of the emulsifier across the epithelium and into the systemic circulation. Permeability enhancers can include, for example, sulfoxides, alcohols, fatty acids and fatty acid esters, polyols, surfactants, terpenes, alkanones, liposomes, ethosomes, cylodextrins. In some embodiments permeability enhancers include, without being limiting, ethanol, glyceryl monoethyl ether, monoglycerides, isopropylmyristate, lauryl alcohol, lauric acid, lauryl lactate, lauryl sulfate, terpinol, menthol, D-limonene, DMSO, polysorbates, N-methylpyrrolidone, polyglycosylated glycerides, Azone®, CPE-215®, NexAct®, SEPA®, and phenyl piperizine.

In some embodiments other methods of administration across an epithelium can be used, for example, iontophoresis, electroporation, sonophoresis, thermal poration, microneedle treatment, and dermabrasion.

In some embodiments, the pharmaceutical formulation is administered so as to achieve circulating levels of at least 50 μM of the emulsifier within 5 minutes after administration. In some embodiments, administration is performed intravenously. In some embodiments, administration occurs intra-arterially. In some embodiments, levels in a range from about 50 μM to about 600 μM are achieved within 5 minutes of administration. In some embodiments, levels in a range from about 100 μM to about 600 μM are achieved within 5 minutes of administration. In some embodiments, levels in a range from about 100 μM to about 300 μM are achieved within 5 minutes of administration.

### Combinations of Emulsifiers and Statins

In some embodiments, a method of treating a patient having, or suspected of having, atherosclerotic plaques can include treatment with an emulsifier as described above, in combination with agents that are effective to lower cholesterol. For example, the class of compounds known as “statins” are effective to lower cholesterol. Statins are inhibitors of HMG-CoA reductase, the rate limiting enzyme in the synthesis of mevalonate, a key intermediate in the synthesis of cholesterol, from acetyl-CoA.

A variety of natural and synthetic statins are known. These include, for example and without being limiting, atorvastatin, cerivastatin, fluvastatin, lovastatin, mevastatin, pitavastatin, pravastatin, rosuvastatin, and simvastatin. Therefore, in some embodiments, a method of treating atherosclerosis, effective to result in a reduction in plaque volume can comprise treatment with an emulsifier as described above effective to achieve a level of the administered emulsifier in the systemic circulation, greater than about 50 uM, in combination with a statin. In some cases, the statin can be administered at a dosage of 20 mg/day; in some cases the statin can be administered at a dosage of 40 mg/day. The statin and emulsifier can be administered concurrently, or sequentially. In some embodiments, the statin and emulsifier can be provided in the same pharmaceutical composition, either as a mixture or in subcompartments of a single dosage form such as a pill, capsule, injectable, or any other suitable form for administration.

In some embodiments, emulsifiers can be administered in combination with a statin and an agent effective to control blood pressure. For example, in some cases emulsifiers can be provided simultaneously, or sequentially, with a statin and a compound like amlodipine.

Emulsifiers, as well as other therapeutic compounds, for example, statins, can be administered by way of a stent. In some embodiments, after an angioplasty procedure, a stent comprising at least one emulsifier as described above, can be placed in a vessel at the site of the angioplasty. The stent is configured to release the emulsifiers in a sustained fashion, such that a local concentration that is effective to dissolve plaques is achieved. The stent can be loaded with one or more emulsifiers, and/or additional therapeutic compounds, and configured to release the therapeutic ingredients over an extended period of time. In some embodiments, the local concentration of emulsifier provided by the stent can be greater than 50 μM. In some embodiments, the local concentration of emulsifier can be in a range from about 50 μM to about 600 μM. In some embodiments, the local concentration of the emulsifier can range from about 100 μM to about 300 μM. Emulsifier eluting stents can be of a balloon expandable design, or self expanding. The stent can also include additional agents effective to dissolve plaque, for example, ionic detergents, nonionic detergents, and zwitterionic detergents. An exemplary list of detergents is provided in International Application PCT/US2007/001214, the entire contents of which are incorporated by reference herein.

In some embodiments, a stent can further comprise enzymes that will digest other components of the plaque (e.g., the fibrous cap), for example proteolytic enzymes such as collagenase, Pronase, Proteinase K, trypsin, chymotrpysin, and other proteases well known to those in the art. Proteases can be selected from classes of proteases including, and without being limiting, serine proteases, threonine proteases, cysteine proteases, aspartic acid proteases, metalloproteases, and glutamic acid proteases. As such, the enzymes listed are understood to be merely exemplary and not exhaustive of the enzymes that can be included in a stent configured for sustained release of emulsifiers. Proteolytic enzymes that are effective to dissolve blood clots, can also be useful in some embodiments of stents that release emulsifiers, in order to prevent, or at least limit, the risk of forming a thrombus at or near the site where the stent is placed in the patient. A stent can also include other therapeutic agents such as antiinflammatory compounds, or compounds that are effective to promote healing of the vessel.

In vitro experiments were performed to test the ability of deoxycholic acid (DCA) to solubilize atherosclerotic plaque material. In these experiments, ex vivo samples of pig artery were bathed in an aqueous solution at two different concentrations of DCA. In the first experiment, samples were treated with 50 mg/mL DCA for successive periods of 30 minutes, at which time the sample was removed from the bathing medium, and the appearance of the plaque examined macroscopically. Early in the treatment, on removal of the sample from the bath a clear, viscous, column of fluid extended from the sample. This column of fluid continued to be apparent when samples were evaluated up to about 4 or 5 hours, after which the fluid column was no longer noted. Without wishing to be held to any one theory of operation, it was concluded that the clear fluid comprised components of the plaque.

After 5 hours of treatment with DCA, macroscopic assessment of plaque size suggested that plaque volume had decreased by about 70%. After 36 hours of exposure all that appeared to remain of plaques were the fibrous cap material and areas of calcification. All core material appeared to have been solubilized.

In a second experiment, atherosclerotic plaque in a sample of pig artery was exposed to a continuous flow of a solution of 0.25 mg/mL DCA, diluted in normal saline (approximately 600 μM DCA). The sample was continuously exposed for a period of 8 days. Macroscopic examination of the sample at this time revealed that most, if not all, of the lipid core of the plaque had been solubilized, and all that remained was the fibrous cap.

In both experiments, treatment with DCA caused no obvious detrimental effects on the vessel itself. In particular, elasticity of the vessel wall appeared unaffected. While not wishing to be held to any one theory of operation, sustained levels of an emulsifier are demonstrated by this example to be effective to produce regression of atherosclerotic plaque, apparently by dissolving the lipid components of the plaque, which once solubilized cross the fibrous cap into the surrounding milieu. In a patient, it is expected that solubilized lipid liberated from plaques by the administered emulsifiers, will be released into the blood stream where they can be metabolized and eliminated from the body by normal physiological routes, for example, by excretion in the bile as free cholesterol, or by conversion to bile acids in the liver.

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. The use of at least two of (i)-(vii) below:
(i) ketoconazole or a pharmaceutically acceptable salt, conjugate, hydrate, solvate, polymorph, or mixture thereof;
(ii) trichloroethylene or a pharmaceutically acceptable salt, conjugate, hydrate, solvate, polymorph, or mixture thereof;
(iii) troglitazone or a pharmaceutically acceptable salt, conjugate, hydrate, solvate, polymorph, or mixture thereof;
(iv) bosentan or a pharmaceutically acceptable salt, conjugate, hydrate, solvate, polymorph, or mixture thereof;
(v) saquinavir or a pharmaceutically acceptable salt, conjugate, hydrate, solvate, polymorph, or mixture thereof;
(vi) ritonavir or a pharmaceutically acceptable salt, conjugate, hydrate, solvate, polymorph, or mixture thereof; and
(vii) efavirenz or a pharmaceutically acceptable salt, conjugate, hydrate, solvate, polymorph, or mixture thereof;
for the manufacture of a medicament useful for treating atherosclerotic plaque.

2. A pharmaceutical formulation, for treating atherosclerosis in a mammal, comprising:
a combination of at least two of (i)-(vii) below:
(i) ketoconazole or a pharmaceutically acceptable salt, conjugate, hydrate, solvate, polymorph, or mixture thereof;
(ii) trichloroethylene or a pharmaceutically acceptable salt, conjugate, hydrate, solvate, polymorph, or mixture thereof;
(iii) troglitazone or a pharmaceutically acceptable salt, conjugate, hydrate, solvate, polymorph, or mixture thereof;
(iv) bosentan or a pharmaceutically acceptable salt, conjugate, hydrate, solvate, polymorph, or mixture thereof;
(v) saquinavir or a pharmaceutically acceptable salt, conjugate, hydrate, solvate, polymorph, or mixture thereof;
(vi) ritonavir or a pharmaceutically acceptable salt, conjugate, hydrate, solvate, polymorph, or mixture thereof;
(vii) efavirenz or a pharmaceutically acceptable salt, conjugate, hydrate, solvate, polymorph, or mixture thereof;
wherein the combination is in an amount effective to result in an amount of increased diversion of a bile acid, from an enterohepatic circulation to the systemic circulation of the mammal, sufficient to result in an amount of emulsification of an atherosclerotic plaque in an artery of the mammal sufficient to result in regression of the plaque.

3. The pharmaceutical formulation of claim 2, wherein the combination comprises ketoconazole or a pharmaceutically acceptable salt, conjugate, hydrate, solvate, polymorph, or mixture thereof, in an amount of 200 mg or greater.

4. The pharmaceutical formulation of claim 2, wherein the combination comprises trichloroethylene or a pharmaceutically acceptable salt, conjugate, hydrate, solvate, polymorph, or mixture thereof, in an amount of 1 mg or greater.

5. The pharmaceutical formulation of claim 2, wherein the combination comprises troglitazone or a pharmaceutically acceptable salt, conjugate, hydrate, solvate, polymorph, or mixture thereof, in an amount of 200 mg or greater.

6. The pharmaceutical formulation of claim 2, wherein the combination comprises bosentan or a pharmaceutically acceptable salt, conjugate, hydrate, solvate, polymorph, or mixture thereof, in an amount of 30 mg or greater.

7. The pharmaceutical formulation of claim 2, wherein the combination comprises saquinavir or a pharmaceutically acceptable salt, conjugate, hydrate, solvate, polymorph, or mixture thereof, in an amount of 1000 mg or greater.

8. The pharmaceutical formulation of claim 2, wherein the combination comprises ritonavir or a pharmaceutically acceptable salt, conjugate, hydrate, solvate, polymorph, or mixture thereof, in an amount of 400 mg or greater.

9. The pharmaceutical formulation of claim 2, wherein the combination comprises efavirenz or a pharmaceutically acceptable salt, conjugate, hydrate, solvate, polymorph, or mixture thereof, in an amount of 200 mg or greater.

10. A method, of treating atherosclerosis in a mammal, comprising administering to a mammal a pharmaceutical formulation in an amount effective to result in an amount of increased diversion of a bile acid, from an enterohepatic circulation to the systemic circulation of the mammal, sufficient to result in an amount of emulsification of an atherosclerotic plaque in an artery of the mammal sufficient to result in regression of the plaque.

11. The method of claim 10, wherein the formulation comprises an active ingredient consisting essentially of at least one of (i)-(vii) below:
(i) ketoconazole or a pharmaceutically acceptable salt, conjugate, hydrate, solvate, polymorph, or mixture thereof;
(ii) trichloroethylene or a pharmaceutically acceptable salt, conjugate, hydrate, solvate, polymorph, or mixture thereof;
(iii) troglitazone or a pharmaceutically acceptable salt, conjugate, hydrate, solvate, polymorph, or mixture thereof;
(iv) bosentan or a pharmaceutically acceptable salt, conjugate, hydrate, solvate, polymorph, or mixture thereof;
(v) saquinavir or a pharmaceutically acceptable salt, conjugate, hydrate, solvate, polymorph, or mixture thereof;
(vi) ritonavir or a pharmaceutically acceptable salt, conjugate, hydrate, solvate, polymorph, or mixture thereof; and
(vii) efavirenz or a pharmaceutically acceptable salt, conjugate, hydrate, solvate, polymorph, or mixture thereof.

12. The method of claim 10, wherein the administering results in a total serum bile acid concentration in the systemic circulation of greater than about 60 μM.

13. The method of claim 10, wherein the administering results in a total serum bile acid concentration in the systemic circulation of about 100 μM to about 300 μM.

14. The method of claim 10, wherein the administering results in a total serum bile acid concentration in the systemic circulation of above about 300 μM.

15. The method of claim 10, wherein the administering results in a total serum bile acid concentration in the systemic circulation of above about 600 μM.

16. The method of claim 10, wherein the bile acid comprises deoxycholic acid.

17. The method of claim 10, wherein the formulation comprises ketoconazole or a pharmaceutically acceptable salt, conjugate, hydrate, solvate, polymorph, or mixture thereof, administered to the mammal at a dose of greater than 600 mg/day.

18. The method of claim 10, wherein the formulation comprises ketoconazole or a pharmaceutically acceptable salt, conjugate, hydrate, solvate, polymorph, or mixture thereof, administered orally to the mammal at a dose of greater than 600 mg/day for at least 7 days.

19. The method of claim 10, wherein the formulation comprises trichloroethylene or a pharmaceutically acceptable salt, conjugate, hydrate, solvate, polymorph, or mixture thereof, administered in a single or divided dose of greater than 135 mg/kg.

20. The method of claim 10, wherein the formulation comprises trichloroethylene or a pharmaceutically acceptable salt, conjugate, hydrate, solvate, polymorph, or mixture thereof, administered in a single or divided dose of greater than 1 mg/kg/day for at least 7 days.

21. The method of claim 10, wherein the formulation comprises troglitazone or a pharmaceutically acceptable salt, conjugate, hydrate, solvate, polymorph, or mixture thereof, administered in a single or divided dose of greater than 30 mg/kg/day.

22. The method of claim 10, wherein the formulation comprises troglitazone or a pharmaceutically acceptable salt, conjugate, hydrate, solvate, polymorph, or mixture thereof, administered in a single or divided dose of greater than 500 mg/day for at least 28 days.

23. The method of claim 10, wherein the formulation comprises bosentan or a pharmaceutically acceptable salt, conjugate, hydrate, solvate, polymorph, or mixture thereof, administered in a single or divided dose of greater than 300 mg/day.

24. The method of claim 10, wherein the formulation comprises saquinavir or a pharmaceutically acceptable salt, conjugate, hydrate, solvate, polymorph, or mixture thereof, administered in a single or divided dose of greater than 3.8 g/day.

25. The method of claim 10, wherein the formulation comprises ritonavir or a pharmaceutically acceptable salt, conjugate, hydrate, solvate, polymorph, or mixture thereof, administered in a single or divided dose of greater than 2 g/day.

26. The method of claim 10, wherein the formulation comprises efavirenz or a pharmaceutically acceptable salt, conjugate, hydrate, solvate, polymorph, or mixture thereof, administered in a single or divided dose of greater than 800 mg/day.

27. The method of claim 10, wherein the formulation comprises at least two of (i)-(vii) below:
(i) ketoconazole or a pharmaceutically acceptable salt, conjugate, hydrate, solvate, polymorph, or mixture thereof;
(ii) trichloroethylene or a pharmaceutically acceptable salt, conjugate, hydrate, solvate, polymorph, or mixture thereof;
(iii) troglitazone or a pharmaceutically acceptable salt, conjugate, hydrate, solvate, polymorph, or mixture thereof;
(iv) bosentan or a pharmaceutically acceptable salt, conjugate, hydrate, solvate, polymorph, or mixture thereof;
(v) saquinavir or a pharmaceutically acceptable salt, conjugate, hydrate, solvate, polymorph, or mixture thereof;
(vi) ritonavir or a pharmaceutically acceptable salt, conjugate, hydrate, solvate, polymorph, or mixture thereof; and
(vii) efavirenz or a pharmaceutically acceptable salt, conjugate, hydrate, solvate, polymorph, or mixture thereof.

28. The method of claim 10, wherein the formulation is administered intravenously.

29. The method of claim 10, wherein the formulation is administered intra-arterially.

30. The method of claim 10, wherein the formulation is administered orally.

31. The method of claim 10, wherein the formulation is administered sublingually.

32. The method of claim 10, wherein the formulation is administered transdermally.

33. The method of claim 10, wherein the formulation is administered via an implantable device.

34. The method of claim 10, wherein the formulation is administered subcutaneously.

35. The method of claim 10, wherein the formulation is administered transmucosally.

36. The method of claim 10, wherein the formulation is administered intramuscularly.
